(19) United States
(12) Patent Application Publication
(10) Pub. No.: US 2004/0033968 A1
(43) Pub. Date: Feb. 19, 2004

(54) Optimal volume of intra-vaginal semisolid dosage forms for treating vaginal infections

(76) Inventors: Shun Y. Lin, Plainsboro, NJ (US); Ying Sun, Belle Mead, NJ (US); Lorraine L. Wearley, Westfield, NJ (US); Brinda Wiita, Princeton, NJ (US); Kalpana J. Patel, West Windsor, NJ (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/222,184

(22) Filed: Aug. 16, 2002

(51) Int. Cl. A61K 38/21; A61K 31/717; A61K 31/7048; A61K 31/496; A61K 31/4196; A61K 31/4168

(52) U.S. Cl. 514/27; 424/85.6; 424/93.4; 514/57; 514/254.07; 514/397; 514/383

(57) ABSTRACT

This invention relates to compositions and methods for treating vaginal infections whereby the patient administers, intravaginally, a volume of semisolid treatment composition, this volume being not greater than 4.7 milliliters, preferably not greater than 4.4 milliliters and more preferably not great than 4 milliliters.

### BACKGROUND OF THE INVENTION

Human vaginal infections, such as vulvovaginal candidiasis (VVC) or bacterial vaginosis (BV), are often treated intra-vaginally with antimicrobial medicament. Several vaginal dosage forms have been developed for over the past decades, such as creams, emulsions, gel, ointment, suppository, tablet, film, capsules and ovules to be used for vaginal treatment. Among these dosage forms, the semisolid dosage form (cream/emulsion, gel, and ointment) is the preferred form for vaginal treatment in the United States.

The human vagina is a fibromuscular tube, the wall of which is covered by vaginal mucosal membrane, and is normally in the relaxed and collapsed stage. The human vagina is approximately three inches (10 cm) in length. The vaginal wall is elastic and muscular, and made of numerous folds (P. Evans, ed. In “The Family Medical Enyclopedia”, Macdonald & Co. Ltd., 1987, NY, page 10). In order to treat infection of the vaginal surface effectively with an intra-vaginal therapy, a sufficient volume of the antimicrobial medicament needs to be applied to cover the whole inner surface of the vaginal cavity, so that no “missed spots” could remain to harbor the pathogenic microbes.

Miconazole nitrate has a fungistatic activity against a variety of pathogenic fungi, including against many Candida species. It has been proven effective in vivo against vulvovaginal candidiasis, as well as many other superficial mycoses, and efficacy has been established with many different formulations. The first available miconazole nitrate vaginal product, MONISTAT® vaginal cream (100 mg), was marketed in the United States in 1974 as a 14-day therapy with an once-a-day dosing regimen. In 1977, MONISTAT® 7 Vaginal Cream 100 mg was approved as a 7-day therapy. Many miconazole nitrate vaginal products (such as MONISTAT® 1 Combination Pack™, MONISTAT® 3 vaginal cream and suppositories) have become available since then for the prescription and over-the-counter treatment of vulvovaginal candidiasis. All cream dosage forms are administered intra-vaginally in a five-gram mass, which is approximately five milliliters in volume.

Terconazole is a synthetic triazole derivative with broad-spectrum antimycotic activity, especially against Candida albicans used for the topical treatment of vulvovaginal candidiasis. Numerous in vitro, in vivo and clinical studies have documented the efficacy of this agent. Earlier terconazole dosage forms were designed as multiple-dose regimens, such as 7-day 0.4% cream (TEPAZOL® 7 Vaginal Cream). As is the case for any other drug therapy, the best choice of treatment is the one that is the most convenient without compromising effectiveness. Many patients have stopped treatment when the symptoms have receded, regardless of whether the infection was completely cured. This may be a contributing factor to the frequent recurrence of vaginal fungal infections. For this reason, both the physicians and the patients desire shorter period of treatment.

To meet the demand for shorter treatment regimens, newer terconazole dosage forms were developed, including 0.8% cream (TERAZOL® 3 Vaginal Cream) and 80mg suppository (TERAZOL® 3 Vaginal Suppository) for three-day treatment. These more recent terconazole formulations have demonstrated effectiveness and comparable cure rate as that of the seven-day treatment. However, it has been found that higher doses of intravaginal terconazole have been associated with increased incidence of adverse reactions such as fever and chills.

Approved vaginal semisolid products containing other active ingredients for treating VVC or BV are also delivered in a 5 gram dosing mass, such as Terazol® (terconazole), Femstat®, Gynazole®-1 (butoconazole nitrate), Vagistat® (tioconazole), Gyne-Lotrimin® (clotrimazole) and Metrogel®-Vaginal (metronidazole). Currently, all the marketed products for VVC or BV treatment in semisolid dosage forms require the application of about 5 grams/ml of a cream, an ointment, or a gel into the vagina. Because the densities of the medicated creams, ointments and gels are usually close to 1 gram/ml, about 5 ml of a semisolid product is used in each application.

Although intra-vaginal treatment for certain medical conditions other than vaginal infections often uses the dose size of a semisolid dosage form less than 5 ml. For example, a dose of 0.5-2 grams is recommended by the manufacturer of Premarin cream containing conjugated estrogens for local treatment of vaginal atrophy. However, for medical conditions of the vagina due to postmenopausal changes, repeated treatments over a rather long period of time (often over months or even years) are often required. The risk of “missed spots” due to a small dose volume is less likely to pose any serious problems for the treatment efficacy because of the non-infectious nature of vaginal atrophy.

Although all these products are considered effective in treating vaginal fungal or bacterial infection, the 5-gram dose usually results in consumer complaints over product leakage/messiness or side effects related to the large dose. There is no information available on the optimal dose volume of semisolid dosage forms for intra-vaginal treatment of vaginal infections. Therefore, it is highly desirable to develop a semisolid product that can minimize leakage/messiness and other side effects, while maximizing the effectiveness of vaginal treatment.

### SUMMARY OF THE INVENTION

We have discovered that, surprisingly, a lower dose volume than the currently-marketed five-ml dose volume of an intravaginal application of a semisolid dosage form to treat vaginal infections results in an unexpectedly greater effectiveness in treating such infections.

While individual vaginal anatomy may vary in terms of its length, width or the number of folds, we have discovered that, surprisingly, there is an optimal deliverable volume of from about 2.3 to about 4.4 ml per application of semisolid dosage form that is preferable. More preferably, a total deliverable volume of 3.4 to 4.1 ml per application should be used for treating vaginal infections including vulvovaginal candidiasis (“VVC”) or bacterial vaginosis (“BV”), and other viral, fungal, bacterial, protozoal and mixed infections caused by such pathogens. Most preferably, about 3.75 ml may be utilized for optimal clinical results, namely, the maximal efficacy and the least adverse effects in treating vaginal infections such as vulvovaginal candidiasis.

Such lower dose volumes generally avoid leakage of medicament from the vaginal cavity which often causes messiness among patients, and consequently, the impairment of patient compliance in administering the treatment.

Clinical results from several intravaginal dosing sizes suggest that, at the intravaginal dose volume sizes of this invention, substantially all the requisite vaginal mucosal surface of an adult human vagina can be covered by a semisolid antifungal preparation without any uncovered “dead spots” in vagina for pathogenic fungi. For certain intravaginal drug products such as terconazole-containing vaginal cream, a reduction in applied dose would lead to improved drug safety profile.

In addition to the use of an optimized volume of a semisolid antimicrobial medicament to treat vaginal infection via intravaginal application, a small volume of the semisolid preparation can be applied topically to the skin of the vulva region, in order to completely eliminate any skin-bound microbial pathogens in the vicinity of the vagina to prevent re-infection and to provide external symptom relief.

### DETAILED DESCRIPTION OF THE PREFERRED EMBODIMENTS

Thus, the compositions of this invention relate to a semisolid vaginal treatment composition containing an active ingredient and a pharmaceutically acceptable carrier, said composition having a unit dose volume of not greater than about 4.7 milliliters. Preferably, said unit dose volume is not greater than about 4.4 milliliters. More preferably, said unit dose volume is not greater than about 4 milliliters.

Preferably, the unit dose volume is from about 0.5 milliliters to about 4.7 milliliters. More preferably, the unit dose volume is from about 2.5 milliliters to about 4.4 milliliters. Most preferably, the unit dose volume is from about 3.5 milliliters to about 4 milliliters.

The compositions of this invention may have active ingredient is selected from the following: an antifungal compound, an antibacterial compound, a moisturizing compound, an antiviral compound and the like or a combination thereof.

Antifungal compositions according to this invention may preferably contain antifungal compounds which are imidazole compounds. More preferably, such antifungal compounds may be selected from the following: fluconazole, tinidazole, secnidazole, miconazole nitrate, econazole, metronidazole, itraconazole, terconazole, posaconazole, ravuconazole, ketoconazole, clotrimazole, saperconazole, fenticonazole, sertaconzaole, butaconazole, tioconazole, cyclopirox and the like and their pharmaceutically acceptable salts or esters or a combination thereof. Most preferably, said antifungal is either miconazole nitrate or terconazole or a combination thereof.

Antibacterial compositions according to this invention may contain antibacterial compounds selected from the following: metronidazole, tinidazole, secnidazole, clindamycin, ornidazole, sodium polystyrene sulfate, sodium cellulose sulfate, vaginal acidifying/buffering agents and the like or a combination thereof. Most preferably, said antibacterial is metronidazole.

Antiviral ingredients include immunomodulators and the like. More preferably, such ingredients may be selected from imiquimod and its derivatives, podofilox, podophyllin, interferon alpha, acyclovir, famcyclovir, valcyclovir, reticulos and cidofovir, a combination thereof and the like.

Antiprotozoal ingredients may include metronidazole and tinidazole, a combination thereof and the like. Probiotic ingredients may be selected from probiotic organisms, including Lactobacillus and Bifidobacterium species, preferably L. rhamnosus, L. acidophilus, L. fermentum, L. casei, L. reuteri, L. crispatus, L. plantarum, L. paracasei, L. jensenii, L. gasseri, L. cellobiosis, L. brevis, L. delbrueckii, L. helveticus, L. salivarius, L. collinoides, L. buchneri, L. rogosal, L. bifidum, B. bifidum, B. breve, B. adolescetis or B. longum.

Buffering agents that may be used in the compositions according to this invention include any physiologically acceptable organic acid and its corresponding salt, either liquid or solid, depending upon the desired form of application. Preferably, such buffers have a pKa from about pH 3 to about pH 5. Buffers that may be useful in the compositions and methods of this invention include, but are not limited to, acetic, fumaric, lactic, citric, propionic, lactic, malic, succinic, gluconic, ascorbic, tartaric acids and the like. Polymers with ionizable functional groups, including, for example, a carboxylic acid or an amine group, and a buffering capacity may also be used as polymeric buffers according to this invention. Examples of polymeric buffers preferably used in the compositions and methods of this invention include Carbomer® or Carbopol®, available commercially from B.F. Goodrich Co., Akron, Ohio, and carboxymethyl celluloses. Virtually any pharmaceutically acceptable buffer system that achieve a pH in the preferred range for topical applications may be used in the compositions and methods of this invention.

Moisturizing compositions according to this invention may contain moisturizing compounds selected from the following: water, glycerin, and the like or a combination thereof.

This invention also relates to a method of treating a vaginal infection wherein the composition is applied intravaginally to a patient suffering from said infection a volume of a semisolid treatment composition comprising an active ingredient and a pharmaceutically acceptable carrier, said volume being not greater than 4.7 milliliters. Preferably, said unit dose volume is not greater than about 4.4 milliliters. More preferably, said unit dose volume is not greater than about 4 milliliters. Preferably, the unit dose volume is from about 0.5 milliliters to about 4.7 milliliters. More preferably, the unit dose volume is from about 2.5 milliliters to about 4.4 milliliters. Most preferably, the unit dose volume is from about 3.5 milliliters to about 4 milliliters.

A semisolid dosage form according to the present invention is well known in the art. The term “semisolid” implies a unique type of Theological behavior as described by G. Flynn in “Modern Pharmaceutics”, (G. S. Banker and C. T. Rhodes, ed. Marcel Dekker, Inc., New York, 1979, pages 299-303). As a class, such systems are plastic in behavior, namely, they retain their shape until acted upon by an outside force, in which case they deform and the deformations are permanent. This particular property allows semisolids to be mechanically spread uniformly over a surface where they cling as nonmobile film. The semisolid systems according to the invention include creams/lotions (oil-in-water and water-in-oil), ointment, aqueous and non-aqueous gels, pastes, foams and the like.

While individual vaginal anatomy may vary in terms of its length, width or the number of folds, it has been discovered that, surprisingly, there is a certain dose volume range of a semisolid dose to be used to achieve the optimal efficacy for intravaginal treatment of infections. This invention provides clinical evidence to show that the optimal dose volume range is greater than about a 2.5-milliliter intra-vaginal dose volume, but lower than the 5 milliliter size typically used in all commercial available vaginal semisolid dosage products for treating VVC or BV. According to the present invention, the preferred dose volume of a semisolid dosage form for intravaginal application ranges from about 0.5 ml to about 4.7 ml, more preferably, between about 2.5 ml to about 4.4 ml, and most preferably, between from about 3.5 ml to about 4 ml.

When the dose volume of an intravaginal semisolid dosage form is low, (e.g., between 0.5 ml and 2.0 ml), the semisolid dosage form should be moderately hypertonic in nature, with its tonicity preferably between about 340 mOsm/L and about 1200 mOsm/L), more preferably between about 450 mOsm/L and about 900 mOsm/L, and most preferably between about 600 mOsm/L and about 800 mOsm/L. The hypertonic semisolid dosage forms are preferably to be oil-in-water cream, hydrophilic gel and microemulsion, containing hygroscopic excipients such as ions, glycols, glycerol, polyethylene glycols and polypropylene glycols of various molecular weights, saccharides and polysaccharides including various sugars, natural and synthetic cellulose gums and gelling agents (e.g., hydroxymethylcellulose, hydroxyethylcellulose, hydroxypropylcellulose, carboxyhydroxymethylcellulose, methylcellulose). Upon applied into the vagina, the hypertonic semisolid dosage form of small volume will rapidly pick up moisture from vaginal fluid to expand its volume to ensure a complete coverage of the infected vaginal mucosal wall.

The typical treatment duration of intra-vaginal therapy for VVC or BV is often less than a week, such as the 3-day once-a-day therapy, or the 1-day single dose therapy. The risk is high for the survived pathogenic microbes on those “missed spots” to thrive once the medicament is removed by the vaginal mucosa cell exfoliation (the vaginal mucosa turnover rate is approximately 3-4 days). Therefore, using the optimal dose size discovered by the present invention will improve patient compliance by eliminating messy leakage of the semisolid medicament from the vagina. More importantly, it will also increase treatment efficacy by avoiding intentional removal of the medicament (due to cleaning the vulvovaginal area by the patients) or unintentional removal of the medicament (adsorption and/or absorption by the undergarment) when a standard dose of 5 grams, or approximately 5 ml, is used intra-vaginally.

The following examples illustrate, but do not limit the scope of the compositions and methods of this invention.

### EXAMPLE 1

Miconazole Nitrate Vaginal Ointments; Clinical Study Design and Results

A single-blind dose ranging efficacy study was performed to compare different doses/volumes of a miconazole nitrate vaginal ointment (TABLE 1) to a commercial VVC product, Gyne-Lotrimin 7 Vaginal Cream. This was a multi-center, single-blind, randomized comparative study to assess the efficacy and safety of same composition with differently deliverable volumes. Patients were seen on admission, treated for one or seven days, and followed up at day 21-30. The studied formulations/regimens are as followed:

Group 1: 2.5 ml of miconazole nitrate vaginal ointment, 1-day single treatment

Group 2: 3.75 ml of miconazole nitrate vaginal ointment, 1-day single treatment

Group 3: 5.0 ml of miconazole nitrate vaginal ointment, 1-day single treatment

Control: Gyne-Lotrimin 7 Vaginal Cream, 7-day once-a-day treatment, 5 ml per application

Since the densities of the miconazole nitrate vaginal ointment and the Gyne-Lotrimin 7 Vaginal Cream are close to 1, the terms of “grams” and “ml” are used interchangeably throughout this document.

This study evaluated the effect of mass delivered or to identified the “a range of tolerable mass”. TABLE 2 shows the study design: by applying the specific volume of the same formulation to the patients in each group, the efficacy and adverse events associated with that particular dose size could be evaluated. In essence, this study allowed us to investigate the relationship between the coverage of vaginal surface and the volume of a semisolid dosage form. A total of 145 patients were valid for efficacy evaluation and a total of 240 patients were valid for safety evaluation.

TABLE 1
Composition of 16% miconazole nitrate ointment used in
EXAMPLE 1.

| Composition | %, w/w |
|---|---|
| Xanthan Gum | 3.00 |
| Sodium Carboxymethylcellulose | 7.00 |
| Silicone Colloidal Dioxide | 1.50 |
| Stearyl Alcohol | 3.50 |
| Polyethylene Glycol 3350 | 8.00 |
| Polyethylene Glycol 400 | 20.00 |
| White Petrolatum | 25.00 |
| Hard Fat | 16.00 |
| Miconazole Nitrate | 16.00 |

TABLE 2
Doses used in three treatment groups

| Treatment | Miconazole nitrate | Miconazole nitrate |
|---|---|---|
| Group | ointment used (ml) | (mg) |
| Group 1 | 2.50 | 400 |
| Group 2 | 3.75 | 600 |
| Group 3 | 5.00 | 800 |

TABLE 3
Efficacy Results

| | | | Microbiologica | | Therapeutic | |
|---|---|---|---|---|---|---|
| Treatment | Clinical cure | | 1 cure | | cure | |
| group | N | % | N | % | N | % |
| Group 1 | 33/46 | 72 | 24/46 | 52 | 20/46 | 44 |
| Group 2 | 25/31 | 81 | 21/31 | 68 | 19/31 | 61 |
| Group 3 | 27/36 | 75 | 22/36 | 61 | 20/36 | 56 |
| Control | 24/32 | 75 | 21/32 | 66 | 18/32 | 56 |

TABLE 4
Safety Results

Adverse Experiences

| Treatment group | N | % |
|---|---|---|
| Group 1 | 33/65 | 51 |
| Group 2 | 24/57 | 42 |
| Group 3 | 26/60 | 43 |
| Control | 31/58 | 53 |

As used herein, “clinical cure” means that no clinical signs or symptoms of VVC were detected upon physical and pelvic examination. “Microbiological cure” means that a culture for candidacies was negative. “Therapeutic cure” means that no additional treatment was indicated for VVC. The overall efficacy of the test groups and the control group were summarized in TABLE 3. Although the therapeutic cure rates for Groups 1, 2 and 3 were relatively close in comparison to the control group (See TABLE 3), the data clearly shows that Group 2 had the best efficacy results in all the cure parameters evaluated, namely, clinical cure, microbiological cure, and therapeutic cure. Group 1 was shown to be the least efficacious among the test groups, implying that 2.5 ml of the dose might not have been able to cover the entire inner vaginal surface to kill off all the fungi efficiently. Surprisingly, Group 2 with the 3.75 ml mass dose demonstrated a better efficacy than the standard 5 gram dose in Group 3. This unexpected finding might be due to the leakage of the medicament from the application site, as often reported with the standard dose of 5 ml.

The safety aspects of the study were assessed based on the adverse experiences reported by the patients during the study period. The most frequently reported adverse experiences were burning, pruritus and irritation of the female genitalia, that can be summarized by primary term as “vulvovaginal discomfort”. However, since itching and burning are two of the most frequent symptoms of VVC, the source of these symptoms are difficult to be differentiated by the patents, namely, whether from the unresolved VVC, or from the treatment itself. This might help to explain the safety results shown in TABLE 4. While adverse experiences from Groups 1 was relatively high probably due to the combination effects of the unresolved VVC symptom and local medicament, the lower adverse experiences reported by the patients in Groups 2 and 3 probably reflected the better treatment efficacy, as set forth in TABLE 3.

This result indicates that the optimal dosing mass for a semisolid dosage form for intra-vaginal treatment of vaginal infections lies somewhere between 2.5 ml and 5 ml, and preferably about 3.75 gram. The clinical results from this study showed that 3.75 ml of the miconazole nitrate ointment provided the best efficacy, possibly by complete coverage of the vaginal surface, leading to reduction of vulvovaginal discomfort, in comparison to other dose size and the control with a commercial product.

What is claimed is:

1. A semisolid vaginal treatment composition comprising an active ingredient and a pharmaceutically acceptable carrier, said composition having a unit dose volume of not greater than about 4.7 milliliters.

2. A composition according to claim 1 wherein said unit dose volume is not greater than about 4.4 milliliters.

3. A composition according to claim 1 wherein said unit dose volume is not greater than about 4 milliliters.

4. A composition according to claim 1 wherein said unit dose volume is from about 0.5 milliliters to about 4.7 milliliters.

5. A composition according to claim 1 wherein said unit dose volume is from about 2.5 milliliters to about 4.4 milliliters.

6. A composition according to claim 1 wherein said unit dose volume is from about 3.5 milliliters to about 4 milliliters.

7. A composition according to claim 1 wherein said active ingredient is selected from the group consisting of: an antifungal compound, an antibacterial compound, a moisturizing compound, an antiviral compound or a combination thereof.

8. A composition according to claim 7 wherein said antifungal compound is selected from the group consisting of: fluconazole, tinidazole, secnidazole, miconazole nitrate, econazole, metronidazole, itraconazole, terconazole, posaconazole, ravuconazole, ketoconazole, clotrimazole, sapirconazole, their salts or esters or a combination thereof.

9. A composition according to claim 7 wherein said antibacterial compound is selected from the group consisting of: metronidazole, tinidazole, secnidazole, clindamycin, ornidazole, sodium polystyrene sulfate, sodium cellulose sulfate, vaginal acidifying/buffering agents or a combination thereof.

10. A composition according to claim 7 wherein said moisturizing compound is selected from the group consisting of: water, glycerin or a combination thereof.

11. A composition according to claim 7 wherein said antiviral compound is selected from the group consisting of: imiquimod and its derivatives, podofilox, podophyllin, interferon alpha, acyclovir, famcyclovir, reticulos and cidofovir, valcyclovir, or a combination thereof.

12. A method of treating a vaginal infection comprising applying intravaginally to a patient suffering from said infection a volume of a semisolid treatment composition comprising an active ingredient and a pharmaceutically acceptable carrier, said volume being not greater than 4.7 milliliters.

13. A method according to claim 12 wherein said volume is not greater than 4.4 milliliters.

14. A method according to claim 12 wherein said volume is not greater than 4 milliliters.

15. A method according to claim 12 wherein said volume is from about 0.5 milliliters to about 4.7 milliliters.

16. A method according to claim 12 wherein said volume is from about 2.5 milliliters to about 4.4 milliliters.

17. A method according to claim 12 wherein said volume is from about 3.5 milliliters to about 4 milliliters.

18. A vaginal treatment composition comprising an active ingredient and a pharmaceutically acceptable carrier comprising a unit dose volume from about 0.5 to about 2 milliliters and having a tonicity from about 340 mOsm/L to about 1200 mOsm/L.

19. A composition according to claim 18 wherein said tonicity is from about 450 mOsm/L to about 900 mOsm/L.

20. A composition according to claim 18 wherein said tonicity is from about 600 mOsm/L to about 800 mOsm/L.

21. A composition according to claim 1 wherein said active ingredient is a probiotic selected from the group consisting of: Lactobacillus and Bifidobacterium species, preferably L. rhamnosus, L. acidophilus, L. fermentum, L. casei, L. reuteri, L. crispatus, L. plantarum, L. paracasei, L. jensenii, L. gasseri, L. cellobiosis, L. brevis, L. delbrueckii, L. helveticus, L. salivarius, L. collinoides, L. buchneri, L. rogosal, L. bifidum, B. bifidum, B. breve, B. adolescetis or B. longum.

22. A composition according to claim 1 wherein said active ingredient is an antiprotozoal selected from the group consisting of: metronidazole, tinidazole or a combination thereof.
